(19) United States
(12) Patent Application Publication
(10) Pub. No.: US 2025/0136917 A1
(43) Pub. Date: May 1, 2025

(54) CELL MODIFICATION SYSTEM AND DEVICE

(71) Applicant: METAL INDUSTRIES RESEARCH & DEVELOPMENT CENTRE, Kaohsiung (TW)

(72) Inventors: Yi-Chia Wu, Kaohsiung (TW); Chun-Chieh Tseng, Kaohsiung (TW); Zong-Yu Xia, Kaohsiung (TW); Tung-Lin Tsai, Tainan (TW); Chun-Ming Chen, Kaohsiung (TW); Chia-Hua Chang, Changhua County (TW); Ling-Zhen Kao, Tainan (TW)

(21) Appl. No.: 18/512,318

(22) Filed: Nov. 17, 2023

(30) Foreign Application Priority Data
Oct. 25, 2023 (TW) 112140846

(51) Int. Cl. C12M 1/42; C12M 1/00; C12M 1/26; C12M 1/36; C12M 3/04; C12N 5/0783

(52) U.S. Cl. CPC C12M 35/04; C12M 23/20; C12M 27/10; C12M 33/10; C12M 35/08; C12M 41/48; C12M 45/05; C12N 5/0636; C12N 2525/00; C12N 2527/00

(57) ABSTRACT

A cell modification system includes a separation module, a modification module, a culture module and a filter module. The separation module is configured to receive a blood sample, separate cells and plasma from the blood sample, and then separate target cells. The modification module is configured to receive the target cells and introduce a modification composite material into the target cells to convert the target cells into modified cells. The culture module is configured to receive and store the modified cells, and monitor a cell proliferation environment to facilitate the activation and proliferation of the modified cells. The filter module is configured to receive the modified cells after proliferating, determine whether the modified cells have been modified successfully, and output the cells that have been modified successfully as a therapeutic product. A cell modification device is also disclosed.

### CROSS REFERENCE TO RELATED APPLICATION

The application claims the benefit of Taiwan application serial No. 112140846, filed on Oct. 25, 2023, and the entire contents of which are incorporated herein by reference.

### BACKGROUND OF THE INVENTION

### 1. Field of the Invention

The present invention relates to a biomedical technique and, more particularly, to a cell modification system and device for simplifying the manufacturing process and preventing cells from being contaminated.

### 2. Description of the Related Art

With the advancement of technology, medical techniques are gradually transitioning from the era of drug treatment to the era of cell therapy. The conventional cell therapy technology involves collecting blood samples from patients and transport them to specialized cell preparation sites. Then, specific types of cells are separated from the samples, and the modification is performed on the collected cells. For example, electroporation is used to temporarily create holes in the cell wall, thereby allowing the composite material to diffuse into the cytoplasm. After the modification, the cells need to be proliferated and cultured for a period of time to reach the number required for medical treatment. Finally, the cell products are transferred back to the medical institution for infusion into the patient for treatment.

The above-mentioned manufacturing process of conventional cell therapeutic products requires transferring cells to different locations between different stages. In addition to time consumption on transferring, it also increases the risk of contamination or improper storage during the transfer process. In addition, the conventional electroporation modification method applies instantaneous high voltage to cells, which may cause damage to cells, resulting in inferior cell therapeutic products or reduced production efficiency.

Thus, it is necessary to improve the conventional cell modification system.

### SUMMARY OF THE INVENTION

To solve the above problem(s), it is an objective of the present invention to provide a cell modification system that can conduct treatments to cells in a single-site station, thereby preventing the cells from being contaminated during transferring among the process stations located at different sites respectively.

It is another objective of the present invention to provide a cell modification system that can connect multiple process modules in series, thereby reducing the time consumed in transferring cells and improving the convenience for operation.

It is still another object of the present invention to provide a cell modification system that can provide cell therapeutic products with better safety and quality, thereby achieving the purpose of customized cell therapy.

It is yet another object of the present invention to provide a cell modification device that can simplify the equipment requirement to improve the convenience for operation.

As used herein, the term “a”, “an” or “one” for describing the number of the elements and members of the present invention is used for convenience, provides the general meaning of the scope of the present invention, and should be interpreted to include one or at least one.

A cell modification system of the present invention includes a separation module, a modification module, a culture module and a filter module. The separation module is configured to receive a blood sample, separate cells and plasma from the blood sample, and then separate target cells. The modification module is communicated with the separation module by a pipeline. The modification module is configured to receive the target cells and introduce a modification composite material into the target cells to convert the target cells into modified cells. The culture module is communicated with the modification module by a pipeline. The culture module is configured to receive and store the modified cells, and monitor a cell proliferation environment to facilitate the activation and proliferation of the modified cells. The filter module is communicated with the culture module by a pipeline. The filter module is configured to receive the modified cells after proliferating, inspect the modified cells to determine whether the modified cells have been modified successfully, and output the modified cells which are determined as modified successfully to be a therapeutic product.

A cell modification device of the present invention includes the above cell modification system, and the separation module, the modification module, the culture module and the filter module are positioned in a housing. The separation module is further communicated with a liquid supply unit configured to apply a blood sample, a culture solution and a modification composite material to the separation module. The filter module is further communicated with a storage unit configured to store cells filtered from the filter module. The liquid supply unit and the storage unit are disposed outside the housing.

Accordingly, the cell modification system and device of the present invention can save the time and cost for transferring cells by integrating the processes of each station for cell modification and connecting the modules with each other through well-sealed pipelines. Moreover, the cell modification system and device of the present invention can also prevent cells from being contaminated during the transfer process, thereby providing the effect of convenience in operation and improving the safety and quality of cell therapeutic products.

In an example, the separation module separates the target cells by using a magnetic activated cell sorting method. Accordingly, the separation module can separate specific target cells in a non-contact manner, thereby providing the effect of preventing from interfering cell activities and reducing damage to cells.

In an example, the modification module has multiple microfluidic channels each including a contraction section and two gradually widened sections located at both ends of the contraction section for allowing a mixed solution of a culture solution, the modification composite material and the target cells to pass through and making the modification composite material diffuse into the target cells. Accordingly, by applying the physical modification as compression and diffusion to the target cells, the effect of preventing the cells from damaging can be achieved.

In an example, a length of the contraction section ranges from 30 microns to 50 microns, and a width of the contraction section ranges from 5 microns to 7 microns, and the two gradually widened sections located at both ends of the contraction section gradually widen to 50 microns. Accordingly, the size and width of the microfluidic channels can be selected according to the size of the cells to be modified, thereby providing the effect of increasing the application range of cell therapy.

In an example, the culture module has a culture tank configured to store the modified cells and a culture solution, and a control unit configured to measure at least one indicator comprising one of the following indicators: a pH value of the culture solution, a carbon dioxide concentration of the culture solution, a surface morphology of the modified cells and a density of the modified cells; and the culture solution is replaced with a new culture solution when a measurement of one of the at least one indicator is abnormal. Accordingly, the culture module can maintain a good cell proliferation environment, thereby providing the effect of improving cell proliferation efficiency and quality.

In an example, the filter module uses an electron microscope to inspect the modified cells, thereby determining the modified cells have been modified successfully in the situation that the modified cells show/are fluorescent. Preferably, the filter module uses an electron microscope to capture an image of the modified cells, thereby determining the modified cells have been modified successfully in the situation that the modified cells show/are fluorescent under inspecting the image. Accordingly, the filter module can filter the cells based on the fluorescence characteristics of the modification materials in the cells, thereby providing the effect of improving the yield of cell therapeutic products.

In an example, the filter module is a bio-optical chip having flow channels and a micro gate, and the filter module controls the micro gate to direct cells that have been successfully modified and cells that have failed to be modified to different flow channels. Accordingly, the filter module can automatically detect and filter the cells, thereby providing the effect of improving filtering efficiency.

In an example, the separation module includes a sorting tank and a mixing tank, and the liquid supply unit is configured to supply the blood sample to the sorting tank and supply the culture solution and the modification composite material to the mixing tank. Accordingly, the separation module can select target cells subjected to modification, and is convenient for the operations of generating modified cells, thereby providing the effect of improving modification efficiency.

In an example, there are two storage units for storing cells that have been successfully modified and cells that have failed to be modified respectively. Accordingly, the cells that have been successfully modified can be used as cell therapeutic products, while the cells that have failed to be modified can be discarded or recycled, thereby providing the effect of improving product yield.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

FIG. 1 illustrates a block diagram of a preferred embodiment of the system of the present invention.

FIG. 2 illustrates a schematic cross-sectional view of the microfluidic channel of the present invention.

FIG. 3 illustrates an enlarged view of the local structure shown in FIG. 2.

FIG. 4 illustrates a schematic structural diagram of a preferred embodiment of the device of the present invention.

### DETAILED DESCRIPTION OF THE INVENTION

In the following description, some preferred embodiments, taken in conjunction with the accompanying drawings, are set forth to provide a thorough understanding of the foregoing and other objects, features, and advantages of the present invention. In addition, if the same symbols are used in different drawings, they are regarded as the same elements, descriptions of which will be omitted.

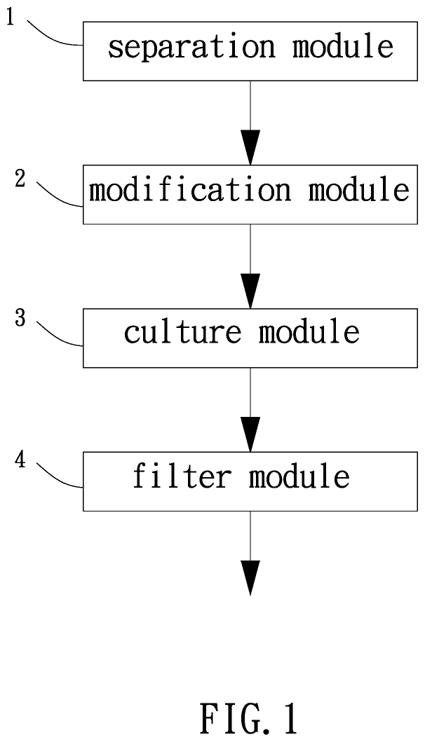

FIG. 1 is a preferred embodiment of a cell modification system of the present invention. The cell modification system comprises a separation module 1 for inputting a blood sample, a modification module 2, a culture module 3 and a filter module 4 for outputting cell products, where the separation module 1, the modification module 2, the culture module 3, and the filter module 4 are connected sequentially by pipeline(s), and the filter module 4 can be also connected to the modification module 2 by a pipeline. These pipelines are preferably medical grade silicone hoses.

The separation module 1 is used to receive the patient's cell sample, which usually enters the separation module 1 in the form of blood. The separation module 1 may include a blood separation unit for separating plasma and cells. Then, it is necessary to further filter and classify the separated cells into such as lymphocytes, stem cells, monocytes, etc. depending on the type and purpose of use. The selected target cells are transferred to the modification module 2 in a sealing manner. Preferably, the separation module 1 separates specific target cells in a non-contact manner, such as electronic control or magnetic control technology. In this embodiment, the separation module 1 employs a magnetic activated cell sorting (MACS) method. The MACS method uses magnetic bead (or microbead) reagents containing specific antibodies to interact with a cell sample, thereby making the selected cells compatible with the reagents and causing only the cells to be selected to carry the magnetic bead(s). When the cell sample passes through a magnetic field, the cells carrying the magnetic beads can be remained to achieve the effect of filtering specific cells. The magnetic beads are biodegradable compositions and do not affect cell activity. In the subsequent cell culture environment, the magnetic beads can rapidly decompose and thus do not interfere with the cell modification process of the present invention.

The modification module 2 compresses and deforms the cells through mechanical deformation, causing a temporary permeability effect on the cell membrane, thereby allowing the modification composite material to enter the cytoplasm through diffusion. In this embodiment, the modification module 2 has several microfluidic channels with a tapered and then enlarged width. The cells are first squeezed and thus create pores when passing through the microfluidic channels, and then generate a pressure difference with the outside when leaving the narrow channel, thereby allowing the modification composite material to enter the cells. The cells are preserved in a culture solution, such that the cells can flow with the culture solution and flow through the microfluidic channel. The modification composite material can be added into the culture solution and then diffuse into the cells when the pores of the cells are opened. In addition, the deformation amount of the cells when being compressed is preferably greater than 30% of the original volume; and the modification composite material can contain epigenetic materials/external genetic materials.

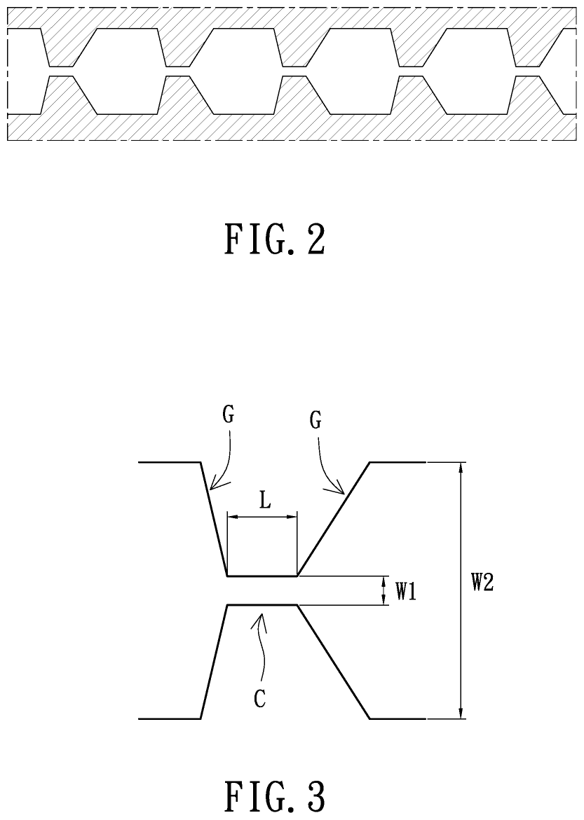

FIGS. 2 and 3 are cross-sectional views of the microfluidic channel stated above. The length L of a contraction section C of the microfluidic channel can be 30 microns to 50 microns, and the width W1 thereof can be 5 microns to 7 microns. The gradually widened sections G located at both ends of the contraction section C gradually widen to a width W2, which may be 50 microns. In addition, as shown in FIG. 2, the microfluidic channel can also be composed of a plurality of contraction sections C connected in series, wherein the number of the contraction sections C connected in series can be 5 to 15. This allows cells passing through the microfluidic channel to repeatedly be compressed, temporarily open pores, and receive external substances to ensure that the cells are successfully modified. The size and width of the microfluidic channels can be selected according to the size of the cells to be modified, and thus the microfluidic channels can be applied to different types of cell therapy.

As shown in FIG. 1, the culture module 3 receives the cells modified by the modification module 2, and then stores the cells in a culture vessel for the culture and proliferation of the modified cells. In this embodiment, specific cytokines or cancer cell markers can be added to the culture vessel to promote cell activation and massive proliferation. The culture module 3 can also maintain a good cell proliferation environment by using a control unit monitoring the growth environment and conditions of the cells. For example, the pH value, carbon dioxide concentration and the like of the culture solution can be measured, such that when the culture solution is not suitable for cell growth, new culture solution can be replaced in time. Alternatively, the cell surface morphology and density can be observed by using a microscope to analyze the cell growth cycle and proliferation conditions. In addition, the culture vessel is preferably capable of centrifugal rotation, and the cells suspended in the culture solution are concentrated to the peripheral area of the culture vessel by centrifugal force, such that the culture solution in the central area of the culture vessel can be replaced with fresh culture solution.

The filter module 4 is used to separate cells that have been successfully modified from cells that have failed to be modified. In this embodiment, the filter module 4 includes a bio-optical chip. The cells cultured by the culture module 3 are provided to the bio-optical chip, and an electron microscope is used to capture cell images to determine whether the modification is successful. In addition, the bio-optical chip may be provided with flow channel(s) and micro gate(s), which can direct cells that have been successfully modified and cells that have failed to be modified to different flow channels. Alternatively, in a situation that the successfully modified cells contain fluorescent components, the filter module 4 can be an optical detection module which uses a light source to illuminate the cells, and then uses a light sensor to detect the fluorescence phenomenon of the cells, such that the intensity and distribution of the fluorescence can be used to determine whether the cells have been successfully modified for providing the effect of filtering.

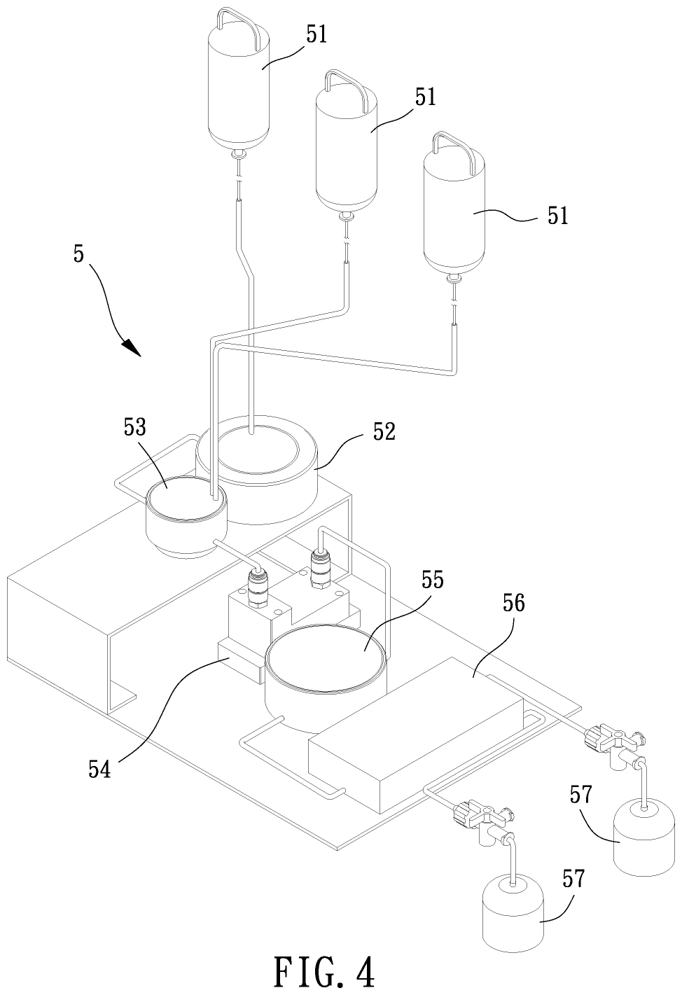

Now referring to FIG. 4, which is a preferred embodiment of the cell modification device 5 of the present invention. The cell modification device 5 includes a liquid supply unit 51, a sorting tank 52, a mixing tank 53, a modification unit 54, a culture tank 55 and a filter tank 56. These components are integrated into the cell modification device 5 and connected in sequence with pipeline(s). In addition, the sorting tank 52, the mixing tank 53, the modification unit 54, the culture tank 55 and the filter tank 56 can be positioned in a housing.

As shown in FIGS. 1 and 4, the cell modification device 5 includes the cell modification system. The separation module 1 includes the sorting tank 52 and the mixing tank 53. The liquid supply unit 51 can be used to supply a blood sample to the sorting tank 52, and supply a culture solution and a modification composite material to the mixing tank 53.

The sorting tank 52 can select the cells to be modified from the original cell solution by using the magnetic bead sorting technology, and transfer the filtering results to the mixing tank 53, such that the cells to be modified, the culture solution and the modification composite material are provided to the modification unit 54 after being mixed in the mixing tank 53.

The modification module 2 includes the modification unit 54, and the culture module 3 includes the culture tank 55. Accordingly, the cell membrane can be temporarily permeabilized by shrinkage deformation, thereby allowing the modification composite material to enter the cell for modification. Then, the modified cells are introduced into and stored in the culture tank 55 for culture and proliferation of the modified cells.

The filter module 4 includes the filter tank 56, and the proliferated cells are provided to the filter tank 56. The filter tank 56 can determine whether the cells have been modified successfully through said bio-optical chip or said optical detection module. The filter tank 56 can be connected to storage units 57, such that the filter tank 56 can direct the cells that have been successfully modified and the cells that have failed to be modified to two different storage units 57 respectively.

In addition, the liquid supply unit 51 and the storage unit 57 can be disposed outside the housing.

It should be noted, based on the above-mentioned configurations among elements/components (such as modules, units and tanks), the cell modification system and device of the present invention can be achieved by coupling those elements with a processor/computer which includes at least one non-transitory memory. The processor is configured to transmit a corresponding control signal to a respective one element when a specific condition of the element is fulfilled. For example, when the filter module 4 uses an electron microscope to inspect the modified cells, and when an intensity of the fluorescent of the modified cells is not lower than a preset threshold value, the processor transmits a control signal to the filter module 4 to make the inspecting modified cells to a storage unit 57 for storing the cells successfully modified. On the contrary, when the intensity of the fluorescent of the modified cells is lower than the preset threshold value, the processor transmits a control signal to the filter module 4 to make the inspecting modified cells to a storage unit 57 for storing the cells failed to be modified. Furthermore, for solution, liquid, blood sample or cells transferring among different elements, a pump, for each element with the transferring function, may be provided and coupled to the processor, so that the transferring function and timing can be controlled by the processor.

In conclusion, the cell modification system and device of the present invention can save the time and cost for transferring cells and prevent cells from being contaminated during the transfer process by integrating the processes of each station for cell modification and connecting the modules with each other through well-sealed pipelines. Moreover, the present invention can prevent cells from being damaged by using a physical modification method of compressing cells to temporarily open pores in the cells, thereby providing the effect of convenience in operation and improving the safety and quality of cell therapeutic products.

Although the invention has been described in detail with reference to its presently preferable embodiments, it will be understood by one of ordinary skill in the art that various modifications can be made without departing from the spirit and the scope of the invention, as set forth in the appended claims.

What is claimed is:

1. A cell modification system, comprising:
a separation module configured to receive a blood sample, separate cells and plasma from the blood sample, and then separate target cells;
a modification module communicated with the separation module by a pipeline, wherein the modification module is configured to receive the target cells and introduce a modification composite material into the target cells to convert the target cells into modified cells;
a culture module communicated with the modification module by a pipeline, wherein the culture module is configured to receive and store the modified cells, and monitor a cell proliferation environment to facilitate the activation and proliferation of the modified cells; and
a filter module communicated with the culture module by a pipeline, wherein the filter module is configured to receive the modified cells after proliferating, inspect the modified cells to determine whether the modified cells have been modified successfully, and output the modified cells which are determined as modified successfully to be a therapeutic product.

2. The cell modification system as claimed in claim 1, wherein the separation module separates the target cells by using a magnetic activated cell sorting method.

3. The cell modification system as claimed in claim 1, wherein the modification module has multiple microfluidic channels each comprising a contraction section and two gradually widened sections located at both ends of the contraction section for allowing a mixed solution of a culture solution, the modification composite material and the target cells to pass through and making the modification composite material diffuse into the target cells.

4. The cell modification system as claimed in claim 3, wherein a length of the contraction section ranges from 30 microns to 50 microns, and a width of the contraction section ranges from 5 microns to 7 microns, and wherein the two gradually widened sections located at both ends of the contraction section gradually widen to 50 microns.

5. The cell modification system as claimed in claim 1, wherein the culture module has a culture tank configured to store the modified cells and a culture solution, and a control unit configured to measure at least one indicator comprising one of the following indicators: a pH value of the culture solution, a carbon dioxide concentration of the culture solution, a surface morphology of the modified cells and a density of the modified cells; and wherein the culture solution is replaced with a new culture solution when a measurement of one of the at least one indicator appears abnormal.

6. The cell modification system as claimed in claim 1, wherein the filter module uses an electron microscope to inspect the modified cells, thereby determining the modified cells have been modified successfully in a situation that the modified cells are fluorescent.

7. The cell modification system as claimed in claim 6, wherein the filter module is a bio-optical chip having flow channels and a micro gate, and wherein the filter module controls the micro gate to direct cells that have been successfully modified and cells that have failed to be modified to different flow channels.

8. A cell modification device, comprising the cell modification system of claim 1, and wherein the separation module, the modification module, the culture module and the filter module are positioned in a housing, wherein:
the separation module is further communicated with a liquid supply unit configured to apply a blood sample, a culture solution and a modification composite material to the separation module, the filter module is further communicated with a storage unit configured to store cells filtered from the filter module, and the liquid supply unit and the storage unit are disposed outside the housing.

9. The cell modification device as claimed in claim 8, wherein the separation module comprises a sorting tank and a mixing tank, and wherein the liquid supply unit is configured to supply the blood sample to the sorting tank and supply the culture solution and the modification composite material to the mixing tank.

10. The cell modification device as claimed in claim 8, wherein there are two storage units for storing cells that have been successfully modified and cells that have failed to be modified respectively.
